(19) United States
(12) Patent Application Publication
(10) Pub. No.: US 2022/0104802 A1
(43) Pub. Date: Apr. 7, 2022

(54) SUTURING APPARATUS USING AUTO-LOADING AND METHOD THEREOF

(71) Applicant: Children's Medical Center Corporation, Boston, MA (US)

(72) Inventor: Kaifeng Liu, Boston, MA (US)

(21) Appl. No.: 17/419,155

(22) PCT Filed: Dec. 2, 2019
(86) PCT No.: PCT/US19/63975

§ 371 Date: Jun. 28, 2021

Related U.S. Application Data
(60) Provisional application No. 62/789,712, filed on Jan. 8, 2019.

(51) Int. Cl. A61B 17/04; A61B 17/062; A61B 17/28

(52) U.S. Cl. CPC A61B 17/0469; A61B 17/0625; A61B 2017/00876; A61B 17/28; A61B 17/0482

(57) ABSTRACT

A suturing system is provided. The suturing system includes a rod having a magnetic tube extending from an end thereof. Additionally, the system includes a magnetic needle having one end attracted into the tube to magnetically engage therewith. The engagement of the needle and the tube is released after the needle is inserted through a material.

### CROSS-REFERENCE TO RELATED APPLICATION

This application claims priority from U.S. patent application Ser. No. 62/789,712 filed on Jan. 8, 2019, entitled the disclosure of which is hereby incorporated herein by reference in it's entirely.

### BACKGROUND

### 1. Technical Field

The present disclosure relates generally to a suturing apparatus using auto-loading and a method thereof, and more particularly, to a suturing apparatus in which a needle is automatically loaded into a holder of the apparatus to simplify the suturing process.

### 2. Description of the Related Art

In general, stitching or suturing, in a medical field, is used to bind pieces of material together. Specifically, suturing is used to stitch together tissue during a surgical procedure. The pieces of tissue will then fuse together during a healing process. A suture needle is typically used to force a suture thread through the layers of tissue to allow the thread to bind the tissue layers together. This suturing process is both time consuming and requires a practitioner to grasp multiple components simultaneously with high accuracy.

A typical suture needles includes a needle tip at one end and a suture connection point at the other end. The length of the suture needle also depends on the type of procedure being performed. For example, a longer suture needle is used for large wounds while a short suture needle is used when access or room for manipulation is limited, such as in small and deep spaces. The tip of the needle is inserted through a material or tissue by applying insertion force to the suture needle. Additional force is then used to guide the remainder of the suture needle through the material until the entire needle is passed therethrough. Since the suture thread is coupled to the suture connection point, the thread is pulled along when the insertion force is applied to the suture needle. The process of inserting the needle with the suture thread through the material or tissue is repeated with another layer of material to bind the materials together.

Developed surgical tools for assisting in this suturing process include instruments that clamp the end of the needle to more easily apply force to the needle through the tissue. In particular, this type of tool requires a user to maintain the clamp grasped against the needle while in use. The clamp is then released once the needle has penetrated through the material or tissue. This instrument, however, requires particular user positioning in order to grasp the needle and also requires continuous manipulation of the clamp. When the needle is to be inserted in a small space, as discussed above, such positioning may be difficult. Additionally, during the operation of the clamp, the needle is unable to be continuously maintained in a secure position, thus increasing potential contamination risk if dropped from the clamp.

According to another developed design, a pair of tweezers may include the suture needle between the tips thereof. In particular, a straight double-ended suture needle is moveable between the tips of the tweezers upon engagement of the tweezers. For example, when a user squeezes the tweezer arms together, the suture needle disconnects from one tip and engages with the other tip. The engagement is based on the release of a blade inside the tweezer arm which must capture a groove of the needle and hold the needle within a recess of the arm. When the tweezers are engaged, the other end of the needle enters an opposite recess and another blade must precisely engage with the needle groove to lock the needle in place. The design, however, is complex requiring multiple interworking elements within the tweezer arms to lock the needle end in place, thus increasing risk of error use.

### SUMMARY

The present disclosure provides a suturing apparatus and method that use auto-loading of a needle to automatically attract the needle into a holder of the apparatus handle to simplify the suturing process, decrease the required time for suturing, and more stably and continuously grip the needle when puncturing through a material.

According to one aspect of the present disclosure, a suturing system includes a rod having a tube that extends from an end thereof wherein the tube is magnetic. In addition, the system includes a magnetic needle with one end attracted into the tube to magnetically engage therewith. The engagement of the needle and tube is then released after the needle is inserted through a material. The system may further include a suture that engages with the needle prior to insertion of the needle through the material.

Further, the tube may extend perpendicularly from the end of the rod. Alternately, the tube may be attached to the rod at a variable angle. The tube may also be detachable from the rod. The tube includes a closed end to secure the needle therein. A proximal end of the needle abuts the closed end of the tube when attracted into the tube. Accordingly, the needle movement is restricted by an inner wall of the tube. An inner diameter of the tube is thus minimally greater than an outer diameter of the needle.

According to another aspect of the present disclosure, a suturing system may include a rod having a channel that extends from an end thereof, wherein one surface of the channel is magnetic. The system may further include a magnetic needle that engages with the magnetic surface of the channel. The engagement of the needle and the magnetic surface of the channel is released after the needled is inserted through a material. The channel may be curved to correspond to a curved shape of the needle.

In addition, a proximal end of the needle abuts a closed end of the channel when the needle is engaged with the magnetic surface of the channel. The magnetic surface of the channel may be a bottom surface. The side surfaces of the channel may be non-magnetic while preventing lateral movement of the needle. The channel may be attached to the rod at a variable angle and may also be detachable from the rod.

According to the exemplary embodiments discussed above, the rod may be an arm of a pair of tweezers or forceps. Additionally, the magnetic surface of the channel, the magnetic tube, and the magnetic needle may each be a permanent magnetic, electromagnet, or a combination of both. Additionally, the magnetic strength and direction of each may be adjustable.

According to yet another aspect of the present disclosure, a method of operating a suturing system may include disposing a magnetic needle near a magnetic holder that extends from an end of a rod of the suturing system to attract the needle into the holder. The holder may be a tube or a channel. The method may further include inserting the needle through a material after the needle is inserted into the holder and then releasing the engagement of the needle and the holder after the needle is inserted through the material. The needle and the holder may then be reengaged after the needle is pulled through the material.

Notably, the present invention is not limited to the combination of the suturing apparatus element as listed above and may be assembled in any combination of the elements as described herein.

Other aspects of the disclosure are disclosed infra.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 illustrates a suturing system having a tubular holder according to an exemplary embodiment of the present disclosure;

FIGS. 2A-2B illustrate engagement of a needle to a holder of the suturing system according to an exemplary embodiment of the present disclosure;

FIG. 3 illustrates puncturing of the needle through a material according to an exemplary embodiment of the present disclosure;

FIG. 4 illustrates a suturing system having a channel according to an exemplary embodiment of the present disclosure;

FIGS. 5A-5B illustrate engagement of a needle to the channel of the suturing system according to an exemplary embodiment of the present disclosure;

FIG. 6 illustrates puncturing of the needle through a material according to an exemplary embodiment of the present disclosure;

FIGS. 7A-7B illustrate a needle having a plurality of grooves according to another exemplary embodiment of the present disclosure;

FIG. 8 illustrates a holder channel having protrusions according to another exemplary embodiment of the present disclosure;

FIG. 9 and FIGS. 10A-10B illustrate a suturing system according to another exemplary embodiment of the present disclosure; and

FIGS. 11A-11C illustrate a method of puncturing the needle of FIG. 9 and FIGS. 10A-10B through a material according to another exemplary embodiment of the present disclosure.

It should be understood that the appended drawings are not necessarily to scale, presenting a somewhat simplified representation of various features illustrative of the basic principles of the disclosure. The specific design features of the present disclosure as disclosed herein, including, for example, specific dimensions, orientations, locations, and shapes will be determined in part by the particular intended application and use environment.

### DETAILED DESCRIPTION

The presently disclosed subject matter will be described more fully hereinafter with reference to the accompanying drawings, in which some, but not all embodiments of the inventions are shown. The presently disclosed subject matter may be embodied in many different forms and should not be construed as limited to the embodiments set forth herein; rather these exemplary embodiments are provided so that this disclosure will satisfy applicable legal requirements. Indeed, many modifications and other exemplary embodiments of the presently disclosed subject matter set forth herein will come to mind to one skilled in the art to which the presently disclosed subject matter pertains, having the benefit of the teachings presented in the foregoing descriptions and the associated drawings. Therefore, it is to be understood that the presently disclosed subject matter is not limited to the specific embodiments disclosed and that modifications and other exemplary embodiments are intended to be included within the scope of the appended claims.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

In one aspect, the present disclosure provides a suturing system that uses auto-loading of a needle to engage the needle with an instrument or holder prior to puncturing through a tissue. The suturing system is thus capable of stably holding the needle in place while puncturing through the tissue which simplifies the suturing process, decreases suturing time, and increasing the accuracy thereof. The system is also capable being operated single-handedly thus further simplifying the suturing process. That is, a user is no longer required to grasp a small needle while pushing the needle through a tissue. Instead, the needle is auto-loaded into a holder and is held inside the holder while the needle is pushed through the tissue. This also allows a user to place stitches in typically inaccessible locations.

In surgical applications, the suture of the suturing apparatus may be a suture thread designed for use with organic tissue and may be composed of an absorbable or non-absorbable material. For example, the thread may be composed of an absorbable material such as catgut, polyglycolic acid, polyactic acid, polydioxanone, caprolactone, or the like. Exemplary non-absorbable materials include polypropylene, polyester, nylon, metallic wires, and the like. In some cases, the thread may be coated with a compound that reduces friction during the suturing process, has antibacterial properties, and/or produces a biological reaction in the patient/subject (e.g., acts as an anti-inflammatory, etc.).

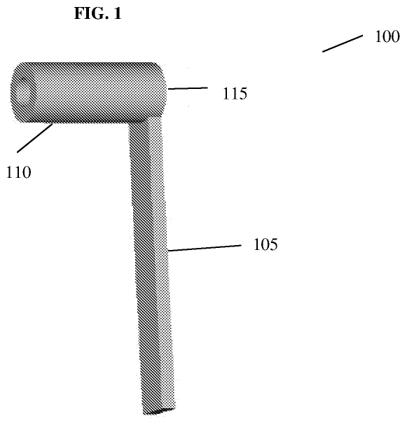

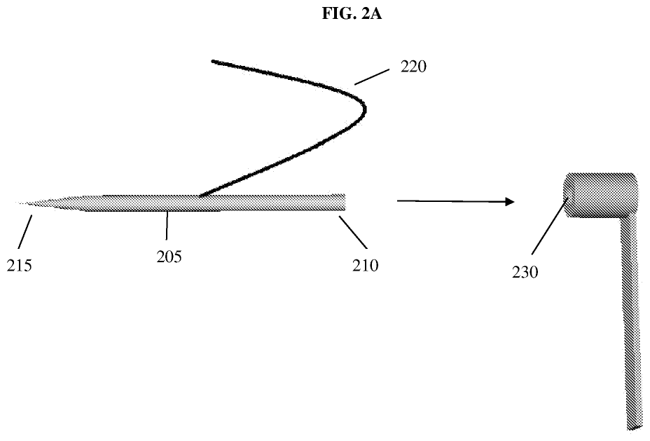

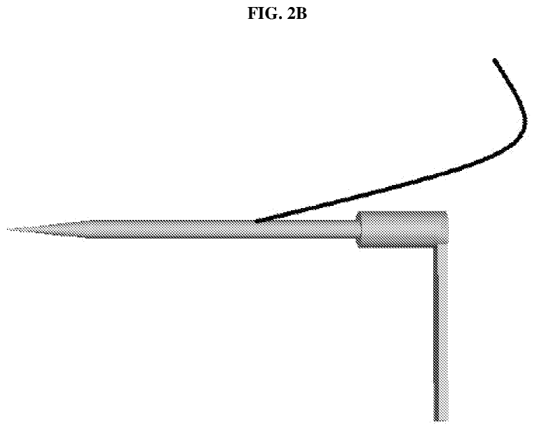

Referring now to FIGS. 1 and 2A, a suturing system 100 is shown according to an exemplary embodiment of the present disclosure. In particular, the suturing system may include a rod 105 having a tube 110 that extends from an end of the rod 105. The tube 110 may have a closed end 115. Additionally, the system may include a magnetic needle 205 having one end (e.g., a proximal end) 210 attracted into the tube 110 to magnetically engage therewith. A second end 215 of the needle 205 may be formed to puncture through a material (e.g., tissue).

As shown by the arrow in FIG. 2A, the proximal end 210 of the needle 205 may be attracted into the tube 110 due to the magnetic force therebetween. Thus, when the needle 205 is merely disposed near the tube 110, the needle 205 will be attracted therein. In particular, when inserted into the needle 205, the proximal end 210 of the needle 205 abuts the closed end 115 of the tube 110.

The figures illustrate the tube extending perpendicularly from the rod, however, the present disclosure is not limited thereto. The tube may be attached to the rod at a variable angle. Additionally, the tube may be detachable from the rod. This facilitates the ability to use the system in typically unreachable or small areas. For example, a user is not required to physically reposition to attempt to reach a surgical area because the tube may be angled differently based on surgical area location. The rod itself may also be the handle of a variety of surgical instruments such as tweezers, forceps, or the like, but is not limited thereto.

Once the proximal end 210 of the needle 205 is attracted into the tube 110, the inner wall 230 of the tube 110 prevents movement of the needle 205. That is, the inner diameter of the tube 110 may be minimally greater than an outer diameter of the needle 205 to thus restrict the movement of the needle once inserted into the tube. This advantageously holds the needle in place while puncturing tissue without requiring the user to grasp the needle in any way. By holding the needle in place, puncturing accuracy is greatly increased as the needle is prevented from wobbling as force is exerted onto the needle to push the needle through the tissue.

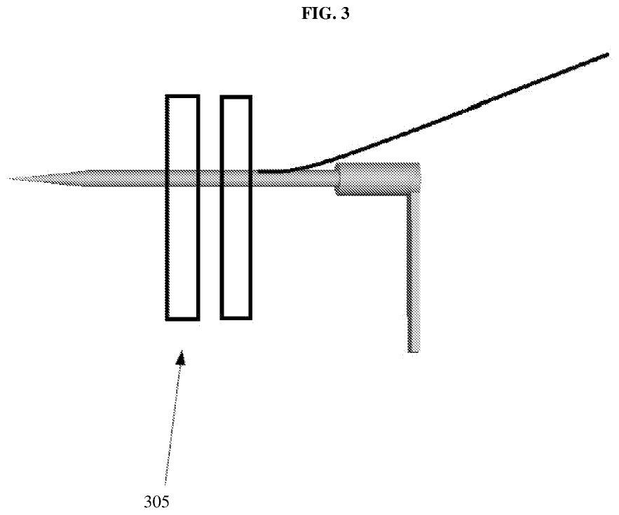

The system may further include a suture 220 that engages with the needle 205 prior to insertion of the needle 205 through a material (e.g., tissue). FIG. 2B illustrates the engagement of the needle 205 with the tube 110 after being attracted thereinto. After such engagement, FIG. 3 illustrates the puncturing of tissue 305 with a needle and suture attached thereto. As shown, the needle 205 together with the suture 220 are penetrated through the tissue 305 to pass the suture 220 therethrough. Once the needle 205 has been inserted through the tissue 305, the engagement of the needle 205 and the tube 110 may be released. The needle may be pulled all the way through the tissue and then may be reengaged with the tube to continue the suturing process. That is, the proximal end 210 of the needle 205 may be attracted to the tube 110 again to engage therewith and then again puncture through the tissue.

The attraction between the magnetic needle 205 and the magnetic tube 110 may be based on a permanent magnet, electromagnet, or a combination thereof with the magnetic strength and direction being adjustable. A further description of this attraction will be discussed below.

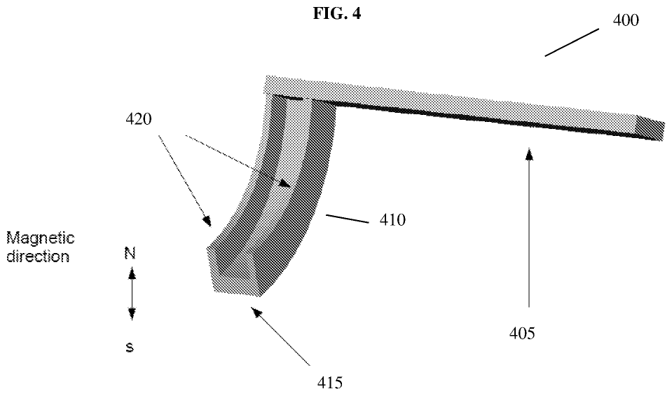

According to another exemplary embodiment and as shown in FIG. 4, a suturing system 400 may include a rod 405 having a channel 410 and a magnetic needle. In particular, the channel 410 may include one magnetic surface which may be a bottom surface 415. However, the present disclosure is not limited thereto and another surface of the channel may be the magnetic surface. FIG. 4 illustrates the magnetic direction of the surface according to an exemplary embodiment of the present disclosure. The side surfaces or side walls 420 of the channel 410 may be non-magnetic. However, the present disclosure is not limited thereto, other surfaces of the channel may be selected to be non-magnetic.

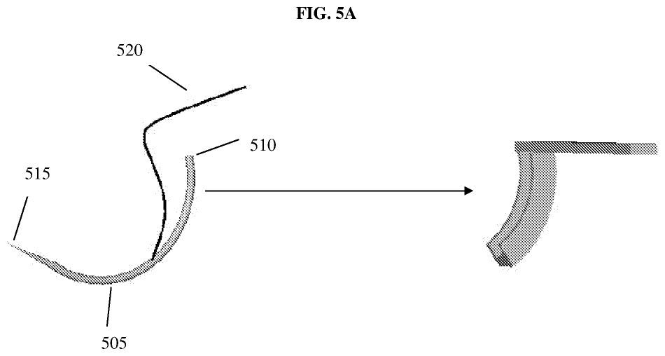

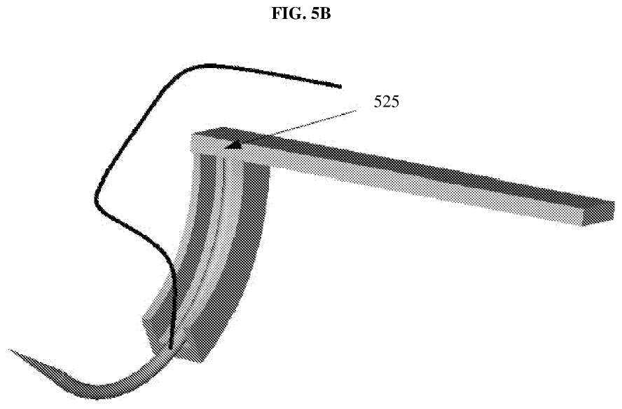

As shown in FIG. 5A, the channel 410 may be curved to correspond to a curved shape of the magnetic needle 505. As further shown by the arrow in FIG. 5A, the corresponding shape of the components, allows the needle 505 to engage with the magnetic surface 415 of the channel 410. During this engagement, a proximal end 510 of the needle 505 may abut a closed end 525 of the channel 410 as shown in FIG. 5B. Once engaged, the side surfaces 420 of the channel 410 may prevent or restrict the lateral movement of the needle 505. This movement restriction advantageously locks the needle in place while pushing the needle through a material. The channel 410 may also be detachable from the rod 405 and may be attached to the rod 405 at a variable angle. The ability to connect the channel and rod at different angles increases the operability of the system by being capable of stably performing a suturing process in confined areas.

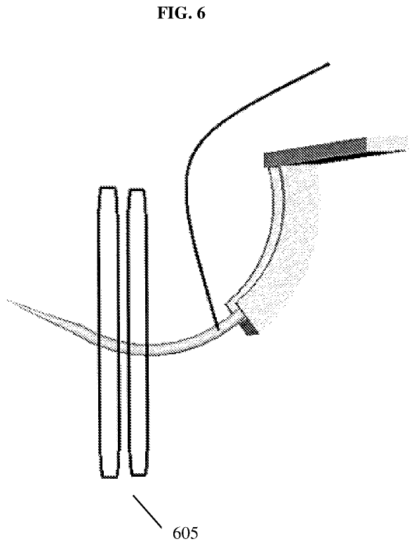

Furthermore, the other end 515 of the needle 505 may be formed to puncture through the material such as tissue. As shown in FIG. 6, the other end 515 of the needle 505 (e.g., the penetrating end, the pointed end, etc.) may be pushed through tissue 605 or any other material.

A suture 520 attached or otherwise engaged with the needle 505 may then pass through the tissue together with the needle 505. After the needle 505 is inserted through the tissue 605, the engagement of the needle 505 and the magnetic surface 415 of the channel 410 may be released to thus allow the needle 505 to be entirely pulled through the tissue 605. The needle 505 and the channel 410 may then be reengaged to continue the suturing process as similarly discussed above.

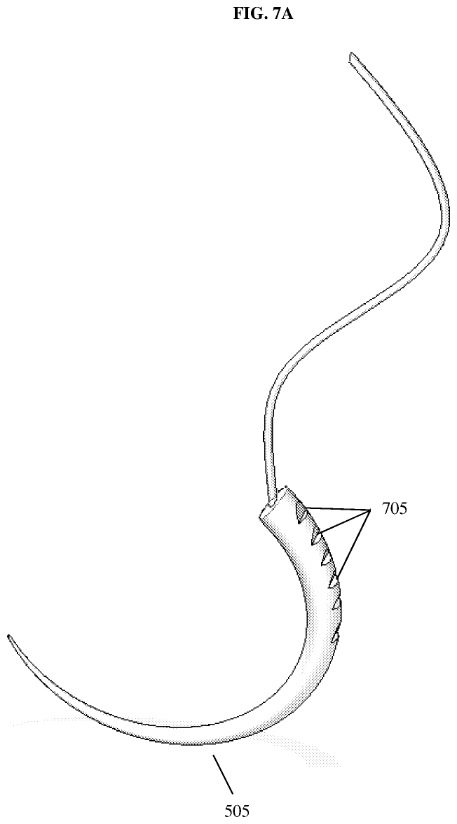

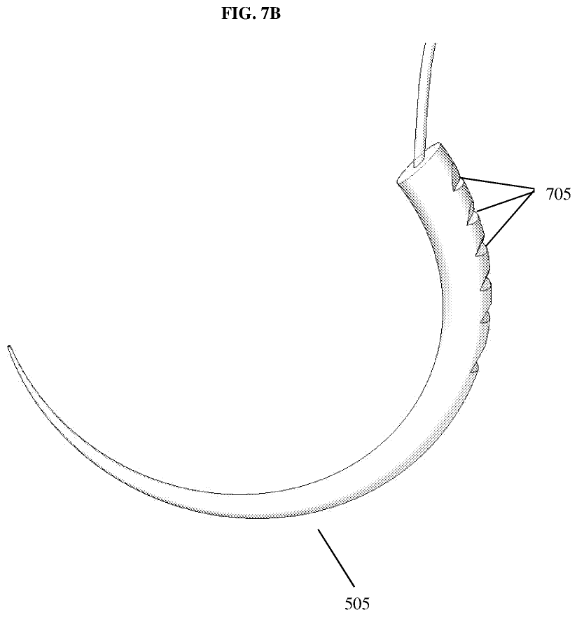

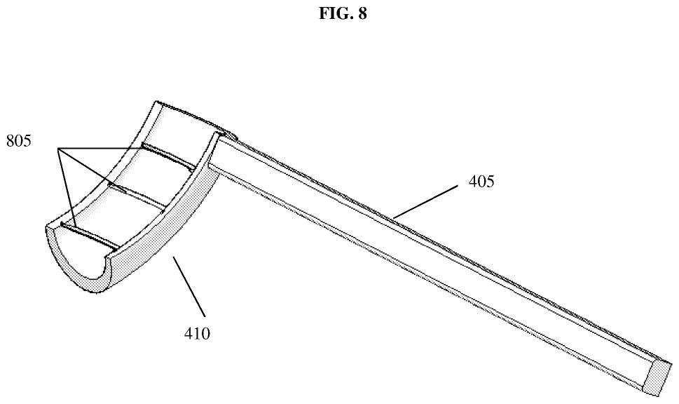

FIGS. 7A-7B and 8 illustrate a suturing system according to another exemplary embodiment. In particular, as shown in FIGS. 7A-7B, the magnetic needle 505 may include a plurality of grooves 705 or dents formed along at least a portion of the outer surface of the needle 505. These grooves provide resistance in one direction to avoid backward movement of the needle. The grooves may be formed in varying lengths and also in varying depths. The spacing between each groove may also be the same or varying. For example, the depth of the grooves may increase in a direction towards the tip of the needle. Additionally, the grooves may be formed along the entire surface of the needle or only a portion thereof.

As shown in FIG. 8, the channel 410 of the suturing system rod 405 may include a plurality of protrusions 805. The protrusions 805 may be formed along the inner surface of the channel and may be formed at varying angles and heights. Thus, when the needle 505 is connected to the channel 410 by the magnetic attraction therebetween, the protrusions 805 are slotted into the grooves 705. That is, the protrusions 805 interlock with the grooves 705 of the needle 505. Accordingly, the contact between the needle 505 and the channel 410 furthers the strength in holding the needle within the channel.

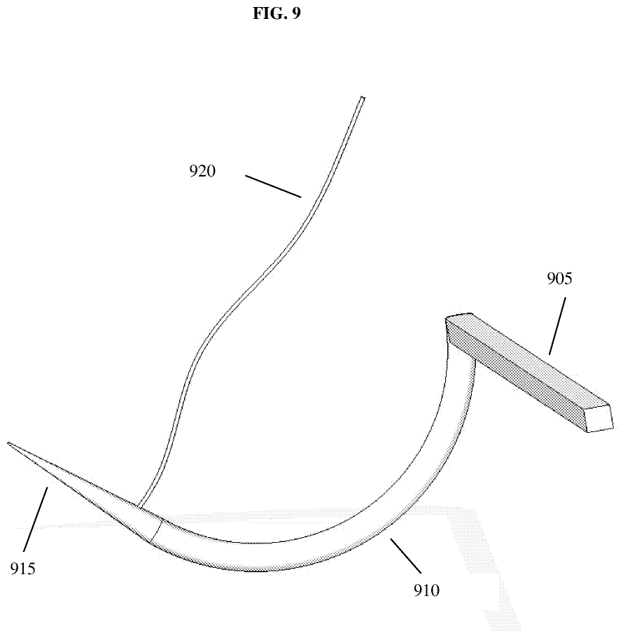

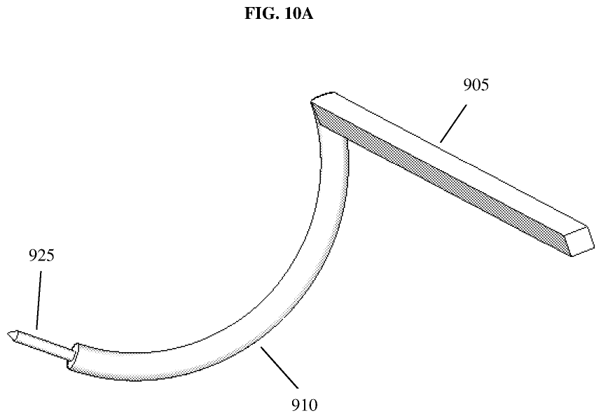

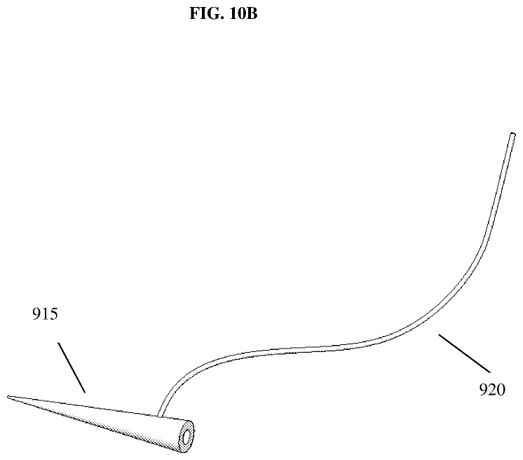

According to another exemplary embodiment of the present disclosure, the rod of the suturing system may be formed integrally with the needle thus eliminating the channel. For example, as shown in FIG. 9, a handle 905 may be formed integrally with the needle 910. A removable needle tip 915 may be attached to the distal end of the needle 910. The suture 920 is connected to the needle tip 915 in this exemplary embodiment. The needle tip 915 may be magnetically connected to the needle 910 (e.g., needle body). As shown in FIG. 10A, the distal end of the needle may be formed with a tip 925 (e.g., needle body tip). The tip 925 may be covered by the needle tip 915. That is, as shown in FIG. 10B, the needle tip 915 may be hollow and the tip 925 may be accommodated or inserted into the needle tip 915 when engaged with the needle body 910.

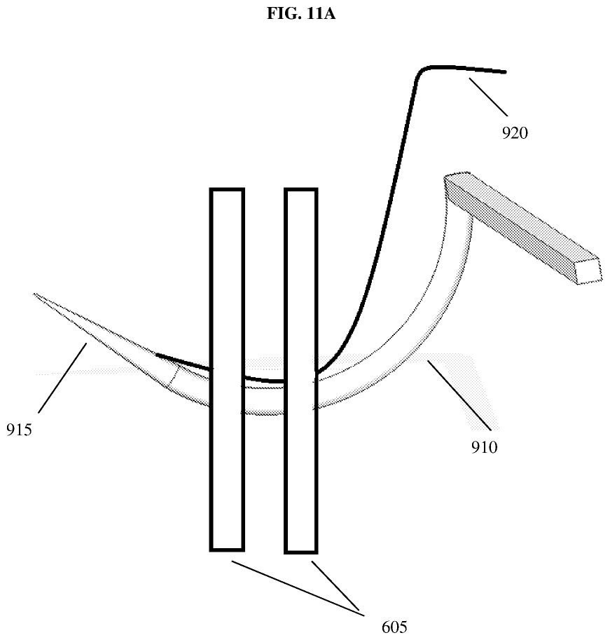

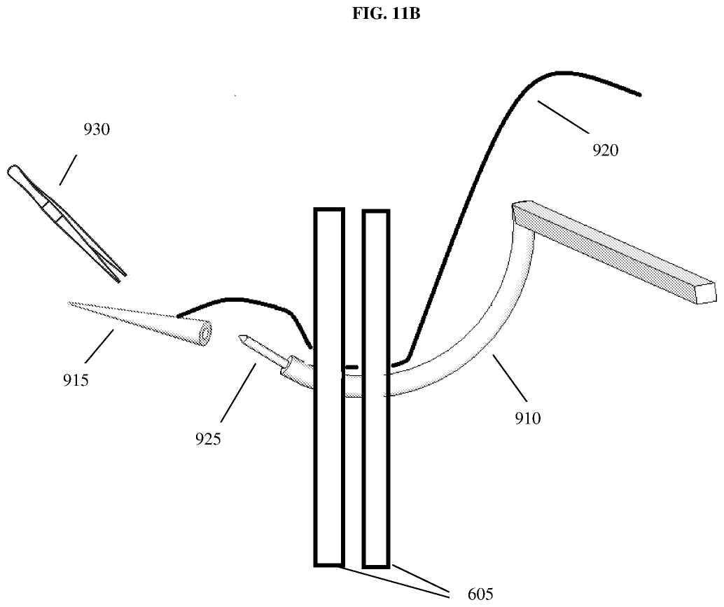

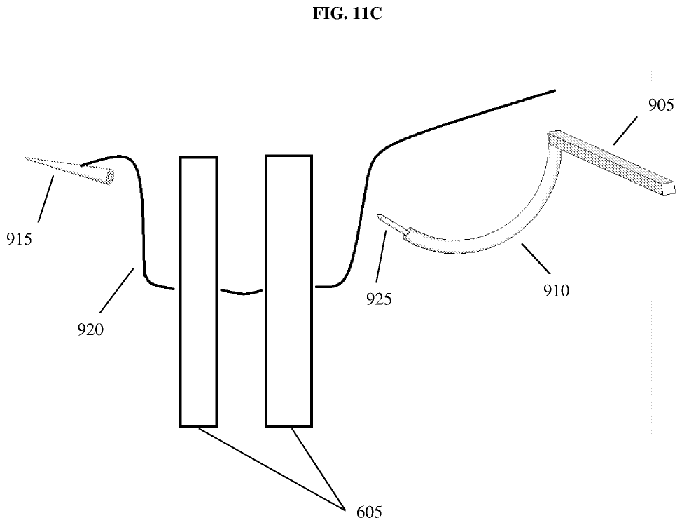

FIGS. 11A-11C illustrate a suturing process with the system of FIGS. 9 and 10A-10B. As first shown in FIG. 11A, the needle 910 may be inserted through tissue 605 with the needle tip 915 engaged at a distal end thereof. Thus, the suture 920 passes through the tissue 605 by being connected to the needle tip 915. As shown in FIG. 11B, after puncturing through the tissue 605, an instrument such as a pair of tweezers 930 may be operated to remove the needle tip 915 from the needle 910 to thus expose the tip 925 (e.g., the body tip). Notably, the present disclosure is not limited to a pair of tweezers and any other instrument may be used. Then, FIG. 11C shows how the needle 910 may be pulled out from the tissue. Accordingly, the tip 925 may reengage with the needle tip 915 to continue the suturing process. This process may be repeated to complete the suturing.

According to another exemplary embodiment, the handle of the suturing system may be eliminated and the suturing may be performed with merely a magnetic needle. In particular, a magnetic field may be generated by a separately provided magnetic generator. The positioning of the magnetic generator adjusts the attraction of the needled. For example, the magnetic generator may be positioned to attract the needle through the tissue, adjust direction of the needle, stop needle movement, and the like in a suturing process.

According to another aspect of the present disclosure, a method of operating a suturing system is provided. In particular, the method may include disposing a magnetic needle near a magnetic holder that extends from an end of a rod of the suturing system. The holder may be a tube, a channel, or any shape and size. That is, in the exemplary embodiments of the present disclosure, the holder is not limited to a tube or channel and may be varied in size or shape. Similarly, the rod is not limited to being a handle and may be an arm of any type of surgical instrument.

Due to the magnetic force of each of the needle and the holder, the needle may be attracted into the holder. Once the needle and holder are engaged to maintain the needle therein, the needle may be inserted through a material such as layers of tissue. After the needle is inserted through the material, the engagement of the needle and the holder may be released. The needle with a suture attached thereto may then be completely passed through the material. To continue the suturing process, the needle and the holder may be reengaged to then puncture the needle through tissue layers again. This process may be repeated until the suturing process is complete.

Notably, the engagement of the needle and holder (e.g., tube, channel, or the like) is not limited to the above-described magnetic connection. The engagement may be via a variety of attractive forces such as mechanical, magnetic, and adhesive techniques that are capable of adjusting the magnetic field strength and direction. As an example, the auto-loading may be accomplished by a technique in which the magnetic field strength and direction changes. That is, the polarity may be reversed after the needle is auto-loaded into the holder and has passed through the tissue to release the engagement between the needle and the holder. The needle and the holder may be composed of oppositely polarized magnets or other materials that exhibit magnetic properties when in presence of a magnetic field. Thus, the needle may exert a magnetic force that magnetically couples with the holder. Then, the needle may be released by reversing the polarity of the magnetic field by creating a magnetic force in the opposite direction.

Alternatively, the suturing system may include power electronics that provide current to the windings of an electromagnet from a power supply via wires, to induce a magnetic field. Power electronics and/or power supply may be housed within the rod of the system. For example, the power supply may include one or more batteries that allow the system to be fully portable. In another example, the power supply may be external to the system. The power electronics operate to control the flow of current to the electromagnet in one or more directions.

For example, the power electronics may have two or three modes of operation. In a dual mode configuration, power electronics may induce an attractive magnetic field when active (e.g., attracting the needle) and remove the magnetic field when deactivated (e.g., disengaging the needle). In a tri-mode configuration, power electronics may induce an attractive magnetic field, induce a repulsive magnetic field by reversing polarity of the electromagnet, or remove the current to the electromagnet completely to deactivate the magnetic field.

The suturing apparatus according to the present disclosure provides numerous advantages. For instance, as discussed herein, the time required to perform suturing may be decreased based on the simplified system and the ability to operate the system with one hand. The system facilitates suturing in traditionally less accessible locations while also providing for more accurate suturing. Additionally, since the needle is fixed within a holder while puncturing a material, the risk of inadvertently puncturing neighboring tissue or dropping the needle is substantially decreased. Notably, the present disclosure is not limited to surgical applications and may be used in any other application requiring connection of materials.

The many features and advantages of the disclosure are apparent from the detailed specification, and thus, it is intended by the appended claims to cover all such features and advantages of the invention which fall within the true spirit and scope of the disclosure. Further, since numerous modifications and variations will readily occur to those skilled in the art, it is not desired to limit the invention to the exact construction and operation illustrated and described, and accordingly, al suitable modifications and equivalents may be resorted to, falling within the scope of the disclosure.

What is claimed is:

1. A suturing system, comprising:
a rod having a tube extending from an end thereof, wherein the tube is magnetic; and
a magnetic needle having one end attracted into the tube to magnetically engage therewith,
wherein the engagement of the needle and the tube is released after the needle is inserted through a material.

2. The suturing system of claim 1, wherein the tube extends perpendicularly from the end of the rod.

3. The suturing system of claim 1, further comprising:
a suture that engages with the needle prior to insertion of the needle through the material.

4. The suturing system of claim 1, wherein the tube includes a closed end to secure the needle therein.

5. The suturing system of claim 4, wherein a proximal end of the needle abuts the closed end of the tube when attracted into the tube.

6. The suturing system of claim 1, wherein needle movement is restricted by an inner wall of the tube.

7. The suturing system of claim 6, wherein an inner diameter of the tube is minimally greater than an outer diameter of the needle.

8. The suturing system of claim 1, wherein the rod is an arm of a pair of tweezers or forceps.

9. The suturing system of claim 1, wherein the tube is detachable from the rod.

10. The suturing system of claim 1, wherein the tube is attached to the rod at a variable angle.

11. The suturing system of claim 1, wherein the magnetic surface of the tube and the magnetic needle are a permanent magnetic, electromagnetic, or a combination thereof.

12. The suturing system of claim 11, wherein the magnetic strength and direction is adjustable.

13. A suturing system, comprising:
a rod having a channel extending from an end thereof, wherein one surface of the channel is magnetic; and
a magnetic needle that engages with the magnetic surface of the channel,
wherein the engagement of the needle and the magnetic surface of the channel is released after the needle is inserted through a material.

14. The suturing system of claim 13, wherein the channel is curved to correspond to a curved shape of the needle.

15. The suturing system of claim 13, wherein a proximal end of the needle abuts a closed end of the channel when the needle is engaged with the magnetic surface of the channel.

16. The suturing system of claim 13, wherein side surfaces of the channel are non-magnetic.

17. The suturing system of claim 16, wherein the side surfaces of the channel prevent lateral movement of the needle.

18. The suturing system of claim 13, wherein the surface of the channel that is magnetic is a bottom surface.

19. The suturing system of claim 13, wherein the rod is an arm of a pair of tweezers or forceps.

20. The suturing system of claim 13, wherein the channel is detachable from the rod.

21. The suturing system of claim 13, wherein the channel is attached to the rod at a variable angle.

22. The suturing system of claim 13, wherein the magnetic surface of the channel and the magnetic needle are a permanent magnetic, electromagnetic, or a combination thereof.

23. The suturing system of claim 22, wherein the magnetic strength and direction is adjustable.

24. A method of operating a suturing system, comprising:
disposing a magnetic needle near a magnetic holder that extends from an end of a rod of the suturing system to attract the needle into the holder;
inserting the needle through a material after the needle is inserted into the holder; and
releasing the engagement of the needle and the holder after the needle is inserted through the material.

25. The method of claim 24, further comprising:
reengaging the needle and the holder after the needle is pulled through the material.
